(19) United States
(12) Patent Application Publication
(10) Pub. No.: US 2013/0084568 A1
(43) Pub. Date: Apr. 4, 2013

(54) Probe, and polymorphism detection method using the same

(71) Applicant: ARKRAY, INC., Kyoto (JP)

(72) Inventors: Kaoru Kurose, Kyoto (JP); Mariko Komori, Kyoto (JP)

(73) Assignee: ARKRAY, INC., Kyoto (JP)

(21) Appl. No.: 13/626,500

(22) Filed: Sep. 25, 2012

(30) Foreign Application Priority Data
Oct. 3, 2011 (JP) 2011-219336
Aug. 30, 2012 (JP) 2012-190186

(51) Int. Cl. C12Q 1/68

(52) U.S. Cl. CPC C12Q 1/6886
USPC 435/6.11; 536/24.31

(57) ABSTRACT

The present disclosure relates to a probe for detecting a polymorphism, a method of detecting a polymorphism, a method of evaluating the efficacy of a drug, and a reagent kit for detecting a polymorphism.

A computer readable text file, entitled “SequenceListing.txt,” created on or about Sep. 24, 2012 with a file size of about 7 kb contains the sequence listing for this application and is hereby incorporated by reference in its entirety.

### BACKGROUND

1. Technical Field

The present invention relates to a probe for detecting a polymorphism, a method of detecting a polymorphism, a method of evaluating the efficacy of a drug, and a reagent kit for detecting a polymorphism.

2. Related Art

A MDR1 gene is a gene encoding P-glycoprotein. P-glycoprotein is expressed in a variety of cells including cancer cells, hepatic cells, intestinal cells and cerebrovascular endothelial cells and plays a role in drug transport.

Further, a mutant-type in which C of the 3435th base on the exon 26 of the MDR1 gene is substituted with T (C3435T mutant-type), a mutant-type in which G of the 2677th base on the exon 21 of the MDR1 gene is substituted with A or T (G2677A/T mutant-type) and a mutant-type in which C of the 1236th base on the exon 12 of the MDR1 gene is substituted with T (C1236T mutant-type) are known to be useful as factors for estimating the prognosis of colon cancer (see, for example, Int. J. Colorectal. Dis., 2010, vol. 25, pp. 1167-1176 (Non-patent Document 1)).

As a method of measuring a gene polymorphism, there is known a method in which, after performing PCR using primers designed to amplify a region containing a base to be measured, the resulting amplification product is cleaved with a restriction enzyme such that the presence or absence of cleavage depends on whether or not a mutation at the base exists and the resultant is then subjected to electrophoresis to detect whether the amplification product has been cleaved or not (PCR-RFLP method).

As an alternative method, there is also known a method in which, after amplifying a region containing a mutation by a PCR method, a nucleic acid probe labeled with a fluorescent dye is hybridized to a target nucleic acid and the decrease in the amount of emission by the fluorescent dye is measured so as to analyze a mutant-type base sequence based on the results of the melting curve analysis (see, for example, Japanese Patent Publication No. 3963422 (Patent Document 1)).

Further, it has been reported that the presence or absence of the C3435T mutant-type of the MDR1 gene can be easily detected with high sensitivity by using a specific nucleic acid probe (see, for example, Japanese Patent Publication No. 4454366 (Patent Document 2)).

In Non-patent Document 1, a PCR-RFLP method was employed as a method of detecting the C3435T mutant-type of the MDR1 gene. In a PCR-RFLP method, it is required that, after PCR is performed, the resulting amplification product be taken out and subjected to a treatment with a restriction enzyme. Therefore, the amplification product may contaminate the subsequent reaction systems, which leads to a false-positive or false-negative result in some cases. In addition, since a treatment with a reaction enzyme is performed after the completion of PCR and the resultant is then subjected to electrophoresis, the time required for detection may become extremely long as well. Furthermore, since the operations of this method are complex, automation thereof is difficult.

Further, in Non-patent Document 1, as a method of detecting the G2677A/T mutant-type and the C1236T mutant-type of the MDR1 gene, a sequencing method is employed. Such a detection method utilizing sequence analysis requires labor and cost since, for example, after performing PCR, it is necessary to perform sequence reactions and then electrophoresis using a sequencer. In addition, since it is required to process the resulting amplification product, the amplification product may contaminate the subsequent reaction systems.

In Patent Document 1, a polymorphism is detected by a method utilizing a nucleic acid probe. However, with regard to the design of the nucleic acid probe, the disclosure offered in Patent Document 1 is merely that the nucleic acid probe is designed in such a manner that, when a quenching probe whose terminal is labeled with a fluorescent dye is hybridized to its target sequence, at least one G-C pair is formed by the plural base pairs of a hybrid generated between the nucleic acid probe and the target sequence at the terminal. Therefore, in the method described in Patent Document 1, it is required to use a nucleic acid probe having an appropriate sequence for each mutant type.

Furthermore, investigation by the present inventors revealed that the quenching probe does not function sufficiently when the GC content is excessively high in the sequence of the nucleic acid probe.

In Patent Document 2, there is disclosed a method of detecting C3435T mutation on the exon 26 of the MDR1 gene; however, the G2677A/T mutant-type and C1236T mutant-type of the MDR1 gene cannot be detected with the nucleic acid probe described in Patent Document 2.

In view of these present circumstances, a further technical development for detecting a polymorphism in bases of the MDR1 gene other than the C3435T mutant-type has been strongly desired.

### SUMMARY OF THE INVENTION

Problems to be Solved by the Invention

An object of the present invention is to provide a polymorphism detection probe which enables C1236T mutation in the exon 12 of the MDR1 gene to be easily detected with high sensitivity; a polymorphism detection method utilizing the probe; a method of evaluating the efficacy of a drug; and a reagent kit for detecting a polymorphism.

### Means for Solving the Problem

The present inventors discovered that, by designing a quenching probe based on a specific region containing C1236T mutation in the exon 12 of the MDR1 gene, C1236T mutation can be detected by performing a melting curve analysis using the quenching probe.

The present invention was made based on such a discovery.
The present invention is as follows.
<1> A probe for detecting a polymorphism in the MDR1 gene, which is a fluorescently labeled oligonucleotide selected from the group consisting of the following P1 and P1′:
(P1) an oligonucleotide having an identity of at least 80% to a sequence complementary to a base sequence of 7 to 38 bases in length including the 395th to the 401st bases of the base sequence indicated in SEQ ID NO:1, wherein a base corresponding to the 395th base is cytosine labeled with a fluorescent dye; and
(P1′) an oligonucleotide which hybridizes under stringent conditions to a complementary strand of a sequence complementary to a base sequence of 7 to 38 bases in length including the 395th to the 401st bases of the base sequence indicated in SEQ ID NO:1, wherein a base corresponding to the 395th base is cytosine labeled with a fluorescent dye.
<2> The probe according to <1>, which is at least one fluorescently labeled oligonucleotide selected from the group consisting of the following P1-1 and P1′-1:
(P1-1) an oligonucleotide having an identity of at least 80% to a sequence complementary to a base sequence of 7 to 38 bases in length including the 395th to the 401st bases of the base sequence indicated in SEQ ID NO:1, wherein a base corresponding to the 395th base is cytosine labeled with a fluorescent dye, the oligonucleotide recognizing a polymorphism at the 401st base of SEQ ID NO:1; and
(P1′-1) an oligonucleotide which hybridizes under stringent conditions to a complementary strand of a sequence complementary to a base sequence of 7 to 38 bases in length including the 395th to the 401st bases of the base sequence indicated in SEQ ID NO:1, wherein a base corresponding to the 395th base is cytosine labeled with a fluorescent dye, the oligonucleotide recognizing a polymorphism at the 401st base of SEQ ID NO:1.
<3> The probe according to <1> or <2>, wherein the above-described P1 fluorescently labeled oligonucleotide or P1′ fluorescently labeled oligonucleotide has a base corresponding to the 395th base labeled with a fluorescent dye at any one of the first to the third positions from the 3′-end.
<4> The probe according to any one of <1> to <3>, wherein the above-described P1 fluorescently labeled oligonucleotide or P1′ fluorescently labeled oligonucleotide has a base corresponding to the 395th base labeled with a fluorescent dye at the 3′-end.
<5> The probe according to any one of <1> to <4>, wherein the above-described P1 fluorescently labeled oligonucleotide or P1′ fluorescently labeled oligonucleotide emits fluorescence when it is not hybridized to a target sequence, and the fluorescence intensity of the P1 fluorescently labeled oligonucleotide or P1′ fluorescently labeled oligonucleotide when hybridized to the target sequence is decreased or increased as compared to when not hybridized to the target sequence.
<6> The probe according to <5>, wherein the above-described P1 fluorescently labeled oligonucleotide or P1′ fluorescently labeled oligonucleotide emits fluorescence when it is not hybridized to a target sequence, and the fluorescence intensity of the P1 fluorescently labeled oligonucleotide or P1′ fluorescently labeled oligonucleotide when hybridized to the target sequence is decreased as compared to when not hybridized to the target sequence.
<7> The probe according to any one of <1> to <6>, wherein the above-described P1 fluorescently labeled oligonucleotide or P1′ fluorescently labeled oligonucleotide has a length of 7 to 28 bases.
<8> The probe according to any one of <1> to <7>, wherein the above-described P1 fluorescently labeled oligonucleotide or P1′ fluorescently labeled oligonucleotide has a length of 7 to 18 bases.
<9> The probe according to any one of <1> to <8>, which is a probe for melting curve analysis.
<10> A method of detecting a polymorphism in the MDR1 gene, the method comprising the processes of:
(I) bringing the probe according to any one of <1> to <9> into contact with a single-stranded nucleic acid contained in a sample to hybridize the above-described fluorescently labeled oligonucleotide to the above-described single-stranded nucleic acid, thereby obtaining a hybrid;
(II) dissociating the above-described hybrid by changing the temperature of the sample containing the hybrid to measure the change in the fluorescence signal caused by dissociation of the hybrid;
(III) determining a Tm value, which is the dissociation temperature of the hybrid, based on the above-described change in the fluorescence signal; and
(IV) based on the above-described Tm value, detecting the presence of a polymorphism of the MDR1 gene on the above-described single-stranded nucleic acid in the above-described sample.
<11> The method according to <10>, which further includes the process of amplifying the nucleic acid prior to or simultaneously with the above-described process (I) of obtaining a hybrid.
<12> The method according to any one of <10> or <11>, which further includes the process of detecting, in the same system, at least either of a polymorphism corresponding to the 256th base of the base sequence indicated in SEQ ID NO:15 or a polymorphism corresponding to the 300th base of the base sequence indicated in SEQ ID NO:16.
<13> The method according to any one of <10> to <12>, which includes the process of detecting, in the same system, a polymorphism corresponding to the 401st base of the base sequence indicated in SEQ ID NO:1, a polymorphism corresponding to the 256th base of the base sequence indicated in SEQ ID NO:15 and a polymorphism corresponding to the 300th base of the base sequence indicated in SEQ ID NO:16.
<14> A method of evaluating efficacy of a drug, which includes the processes of: detecting a polymorphism in the MDR1 gene by the method of detecting a polymorphism according to any one of <10> to <13>; and
   determining tolerance to the drug or the efficacy of the drug based on the presence or absence of detected polymorphism.
<15> A reagent kit for detecting a polymorphism in the MDR1 gene, which includes the probe according to any one of <1> to <9>.
<16> The reagent kit according to <15>, which further includes a primer for amplifying a base sequence containing a region to which the above-described probe hybridizes.
<17> The reagent kit according to <15> or <16>, which further includes a probe for detecting a polymorphism corresponding to the 256th base of the base sequence indicated in SEQ ID NO:1 and a probe for detecting a polymorphism corresponding to the 300th base of the base sequence indicated in SEQ ID NO:16.

### Effects of the Invention

According to the present invention, a polymorphism detection probe which allows C1236T mutation in the exon 12 of the MDR1 gene to be easily detected with high sensitivity; a polymorphism detection method utilizing the probe; a method of evaluating the efficacy of a drug; and a reagent kit for detecting a polymorphism can be provided.

### BRIEF DESCRIPTION OF THE DRAWINGS

FIG. 1A is an example of a melting curve of a nucleic acid mixture, and FIG. 1B is an example of a differential melting curve of a nucleic acid mixture.

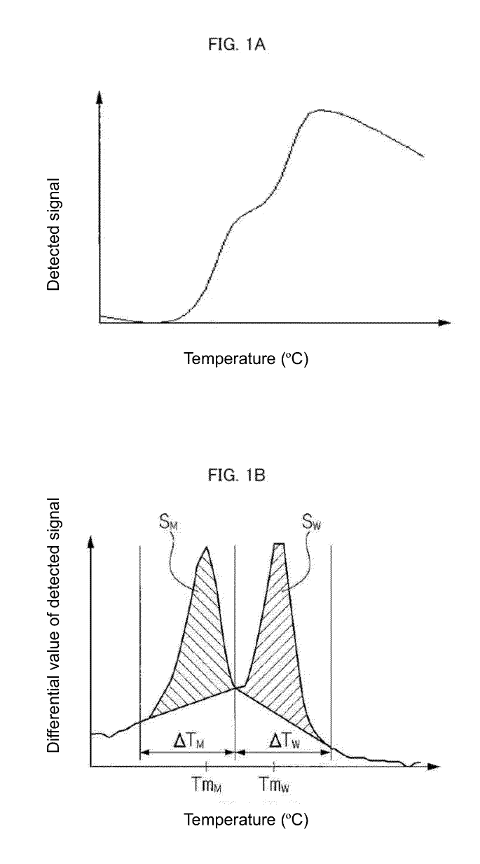

FIG. 2 is differential melting curves of sample according to Example 1 of the present invention.

FIG. 3A to FIG. 3F are differential melting curves of samples according to Example 2 of the present invention.

FIG. 4 is differential melting curves of samples according to Comparative Example 1 of the present invention.

### DETAILED DESCRIPTION OF THE INVENTION

The probe for detecting a polymorphism in the MDR1 gene according to the present invention (hereinafter also simply referred to as “the probe”) is a probe for detecting a polymorphism in the MDR1 gene, the probe including one fluorescently labeled oligonucleotide selected from the group consisting of a P1 fluorescently labeled oligonucleotide and a P1′ fluorescently labeled oligonucleotide:
(P1) an oligonucleotide having an identity of at least 80% to a sequence complementary to a base sequence of 7 to 38 bases in length including the 395th to the 401st bases of the base sequence indicated in SEQ ID NO:1, wherein a base corresponding to the 395th base is cytosine labeled with a fluorescent dye; and
(P1′) an oligonucleotide which hybridizes under stringent conditions to a complementary strand of a sequence complementary to a base sequence of 7 to 38 bases in length including the 395th to the 401st bases of the base sequence indicated in SEQ ID NO:1, wherein a base corresponding to the 395th base is cytosine labeled with a fluorescent dye.
   The method of detecting a polymorphism in the MDR1 gene according to the present invention is a method which includes detecting a polymorphism in the MDR1 gene by using at least one probe for detecting a polymorphism in the MDR1 gene as described above. The method of evaluating the efficacy of a drug according to the present invention is a method which includes detecting a polymorphism in the MDR1 gene by the above-described method of detecting a polymorphism in the MDR1 gene, and evaluating the tolerance to a drug or the efficacy of a drug based on the detected presence or absence of the polymorphism. The reagent kit for detecting a polymorphism according to the present invention is a kit which contains the probe for detecting a polymorphism in the MDR1 gene.

The “MDR1 gene” in the present invention is already known, and the base sequence thereof corresponds to the 4926th to the 214386th bases of Gene ID: 5243, NCBI Accession No. NG_{—}011513. It is noted here that, in the present specification, unless otherwise specified, “the MDR1 gene” means the base sequence corresponding to the exon 12 of the MDR1 gene indicated in SEQ ID NO:1.

The base sequence of SEQ ID NO:1 is a sequence of the 1st to the 801st bases of NCBI dbSNP Accession NO. rs1128503.

In the present invention, the descriptions of the base sequences of the sample nucleic acid in a sample to be detected and the polymorphism detection probe or primer shall also apply to complementary base sequences thereof, respectively, unless otherwise specified. Further, when the description of a particular base sequence is applied to a complementary base sequence thereof, descriptions of base sequences recognized by the particular base sequence in the present invention should be applied provided that the recognition by the particular base sequence should be replaced with recognition by a complementary base sequence of the particular base sequence, within a range of the common general technical knowledge of those skilled in the art.

In the present invention, the term “Tm value” is defined as a temperature at which a double-stranded nucleic acid dissociates (dissociation temperature: Tm), and is generally defined as a temperature at which the absorbance at 260 nm has increased by 50% of the total increase in absorbance resulting from complete dissociation of the double-stranded nucleic acid. More specifically, when a solution containing a double-stranded nucleic acid such as a double-stranded DNA is heated, the absorbance at 260 nm of the double-stranded nucleic acid gradually increases. This is because the hydrogen bonds between both strands of the double-stranded DNA are broken by heating, thereby dissociating the double-stranded DNA into single-stranded DNAs (melting of DNA). When the double-stranded DNA has completely dissociated into single-stranded DNAs, the single-stranded DNAs exhibit an absorbance that is about 1.5 times the absorbance at the time of the initiation of the heating (i.e., the absorbance when the entire DNA is in the form of a double-stranded DNA), which serves as an indicator of the completion of the melting. The Tm value is defined based on this phenomenon.

In the present invention, when the phrase “the first to third bases from the 3′ end” is used in connection to an oligonucleotide sequence, it is assumed that the base at the 3′ end of the oligonucleotide chain is the first base from the 3′ end. Similarly, when the phrase “the first to third bases from the 5′ end” is used in connection to an oligonucleotide sequence, it is assumed that the base at the 5′ end of the oligonucleotide chain is the first base from the 5′ end.

In the present specification, the scope of the term “process” includes not only a discrete process, but also a process that cannot be clearly distinguished from another process as long as the expected effect of the process of interest is achieved.

In the present specification, any numerical range expressed using “to” refers to a range including the numerical values before and after “to” as the minimum and maximum values, respectively.
In a case in which the amount of a component that may be included in the composition is indicated in the present invention, when there are plural substances corresponding to the component in the composition, the indicated amount means the total amount of the plural substances present in the composition, unless specifically stated otherwise.
In the present specification, the term “mutation” refers to a base sequence newly produced by substitution, deletion, overlapping or insertion of a part of a wild-type base sequence. Further, the term “polymorphism” refers to a polymorphism of a base sequence caused by a mutation.
The present invention is described below.

<Probe for Detecting Polymorphism in MDR1 Gene>

The probe for detecting a polymorphism in the MDR1 gene according to the present invention (hereinafter also simply referred to as “the probe”) is a probe for detecting a polymorphism in the MDR1 gene, the probe including one fluorescently labeled oligonucleotide selected from the group consisting of a P1 fluorescently labeled oligonucleotide and a P1′ fluorescently labeled oligonucleotide:
(P1) an oligonucleotide having an identity of at least 80% to a sequence complementary to a base sequence of 7 to 38 bases in length including the 395th to the 401st bases of the base sequence indicated in SEQ ID NO:1, wherein a base corresponding to the 395th base is cytosine labeled with a fluorescent dye; and
(P1′) an oligonucleotide which hybridizes under stringent conditions to a complementary strand of a sequence complementary to a base sequence of 7 to 38 bases in length including the 395th to the 401st bases of the base sequence indicated in SEQ ID NO:1, wherein a base corresponding to the 395th base is cytosine labeled with a fluorescent dye.

In the present invention, the above-described P1 and P1′ fluorescently labeled oligonucleotides are probes capable of detecting a polymorphism at the 401st base of the base sequence indicated in SEQ ID NO:1.

In the wild-type MDR1 gene, the base corresponding to the 401st base of the sequence indicated in SEQ ID NO:1 is C (cytosine); however, in a mutant-type, the C is mutated to T (thymine) (hereinafter, also referred to as “the C1236T mutant-type”) and this base corresponds to the 87296217th base of the 87133179th to the 87342639th bases of the MDR1 gene.

In the present invention, the above-described P1 and P1′ fluorescently labeled oligonucleotides are base sequences complementary to a base sequence including the 395th to the 401st bases of SEQ ID NO:1.

Specifically, in the present invention, a fluorescently labeled oligonucleotide observed with a homology has an identity of at least 80% to a sequence complementary to a base sequence of 7 to 38 bases in length including the 395th to the 401st bases of the base sequence indicated in SEQ ID NO:1. Here, the base corresponding to the 395th base is required to be cytosine.

Further, from the standpoint of the detection sensitivity, the P1 fluorescently labeled oligonucleotide may also exhibit an identity of not less than 85%, not less than 90%, not less than 95%, not less than 96%, not less than 97%, not less than 98% or not less than 99% to a sequence complementary to a base sequence of 7 to 38 bases in length including the 395th to the 401st bases of the base sequence indicated in SEQ ID NO:1.
When the identity between the above-described P1 fluorescently labeled oligonucleotide of the present invention and the base sequence having the same bases as SEQ ID NO:1 except that the base corresponding to the 217th base is C (cytosine) is less than 80%, the detection sensitivity for a sample nucleic acid containing a mutant-type MDR1 gene becomes low.

In the present invention, the above-described P1′ fluorescently labeled oligonucleotide hybridizes under stringent conditions to a complementary strand of a sequence complementary to a base sequence of 7 to 38 bases in length including the 395th to the 401st bases of the base sequence indicated in SEQ ID NO:1. Here, the base corresponding to the 395th base is required to be cytosine.

The hybridization may be carried out according to a known method or a method corresponding thereto, such as a method as described in Molecular Cloning 3rd (J. Sambrook et al., Cold Spring Harbor Lab. Press, 2001). This document is incorporated herein by reference.

The term “stringent conditions” means conditions in which specific hybrids are formed, but non-specific hybrids are not formed. Typical examples of the stringent conditions include, for example, conditions in which the hybridization is carried out at a potassium concentration from about 25 mM to about 50 mM and a magnesium concentration from about 1.0 mM to about 5.0 mM. One example of the conditions of the present invention is conditions in which the hybridization is carried out in Tris-HCl (pH 8.6), 25 mM KCl, and 1.5 mM MgCl₂, but examples of the conditions of the present invention are not limited thereto. Other examples of the stringent conditions are described in Molecular Cloning 3rd (J. Sambrook et al., Cold Spring Harbor Lab. Press, 2001). This document is incorporated herein by reference. Those skilled in the art may readily choose such conditions by changing the hybridization reaction and/or the salt concentration of the hybridization reaction solution.

Furthermore, the P1 or P1′ fluorescently labeled oligonucleotide in the present invention encompasses a fluorescently labeled oligonucleotide having a sequence wherein a base(s) have been inserted to, deleted from and/or substituted in the P1 or P1′ fluorescently labeled oligonucleotide.

The fluorescently labeled oligonucleotide having a sequence wherein a base(s) have been inserted, deleted and/or substituted is not particularly limited, as long as the oligonucleotide exhibits an effect similar to that of the P1 or P1′ fluorescently labeled oligonucleotide; and, in cases where a base(s) have been inserted, deleted and/or substituted, the position(s) of the insertion(s), deletion(s) and/or substitution(s) are not particularly limited. The number of bases that have been inserted, deleted and/or substituted may be, for example, 1 base, or 2 or more bases, such as from 1 base to 10 bases and from 1 base to 5 bases, although varying depending on the total length of the fluorescently labeled oligonucleotide.

The oligonucleotides in the above-described fluorescently labeled oligonucleotide also encompass oligonucleotides as well as modified oligonucleotides.

Examples of a structural unit of the above-described oligonucleotide include ribonucleotides, deoxyribonucleotides and artificial nucleic acids. Examples of the artificial nucleic acids include DNAs, RNAs, LNAs (Locked Nucleic Acids) which are RNA analogues, PNAs (Peptide Nucleic Acids) which are peptide nucleic acids, BNAs (Bridged Nucleic Acids) which are cross-linked nucleic acids, and the like.
The above-described oligonucleotides may be constituted by one or plural types of the structural units described in the above.

The P1 or P1′ fluorescently labeled oligonucleotide of the present invention needs to have a length of from 7 mer to 38 mer. When the P1 or P1′ fluorescently labeled oligonucleotide has a length of 6 mer or less or a length of 39 mer or more, the sensitivity for detecting a polymorphism in the MDR1 gene will be decreased.

In addition, the P1 or P1′ fluorescently labeled oligonucleotide of the present invention may have a length of from 7 mer to 28 mer, or a length of from 7 mer to 18 mer. By using the range of from 7 mer to 28 mer, for example, the detection sensitivity tends to be higher. By varying the base length of the P1 or P1′ fluorescently labeled oligonucleotide, for example, the Tm value, which is a dissociation temperature of a hybrid formed by the P1 or P1′ fluorescently labeled oligonucleotide and its complementary strand (target sequence), may be adjusted to a desired value.

Examples of the base sequence of the P1 or P1′ fluorescently labeled oligonucleotide in the present invention are shown in Table 1 below, but the present invention is not limited to these.

It is noted here that, in Table 1, the base corresponding to the 401st base of SEQ ID NO:1 is indicated with a capital letter. In addition, Table 1 also shows the Tm values of hybrids formed between the respective sample nucleic acids having the same bases as SEQ ID NO:1 except that the base corresponding to the 401st base is T or C and the P1 fluorescently labeled oligonucleotide.
The Tm values were calculated by using MeltCalc© 99 FREE (http://www.meltcalc.com/) and under the set conditions of: Oligoconc. [μM] of 0.2 and Na eq. [mM] of 50.

TABLE 1

| | Length | Tm value | | Δ value between | SEQ |
|---|---|---|---|---|---|
| Sequence (5′→3′) | (mer) | T | C | C and T | ID NO: |
| Acccttc | 7 | 2.8 | −11.9 | 14.7 | — |
| ttcaggttcagAcccttc | 18 | 50 | 41 | 9 | 2 |
| gcaccttcaggttcagAcccttc | 23 | 59 | 53.2 | 5.8 | 3 |
| actctgcaccttcaggttcagAcccttc | 28 | 63.1 | 58.7 | 4.4 | 4 |
| ccgtctgcccactctgcaccttcaggttcagAcccttc | 38 | 71.6 | 68.6 | 2.6 | 5 |
| accagggccaccgtctgcccactctgcaccttcaggttcagAcccttc | 48 | 76.3 | 74.5 | 1.8 | 6 |

In the present invention, the difference between the Tm value when the fluorescently labeled oligonucleotide is hybridized with a sample nucleic acid in which the base corresponding to the 401st base of SEQ ID NO:1 is T and the Tm value when the fluorescently labeled oligonucleotide is hybridized with a sample nucleic acid in which the base corresponding to the 401st base of SEQ ID NO:1 is C is preferably not less than 1.5° C., more preferably not less than 2.0° C., still more preferably not less than 5° C., and yet still more preferably not less than 9.0° C. When the above-described difference in the Tm value is not less than 1.5° C., for example, a mutant-type of the 401st base in SEQ ID NO:1 can be detected with a higher sensitivity.

Examples of a method of increasing the difference in the Tm value include a method by which a probe is allowed to contain a base which mismatches with a base sequence corresponding to a region to which the probe hybridizes. Specific examples include those methods described in Nature Biotech (1997) vol. 15, pp. 331-335 and the like.

Further, the P1 or P1′ fluorescently labeled oligonucleotide of the present invention needs to be labeled with a fluorescent dye at its base corresponding to the 395th base (cytosine).

In the P1 or P1′ fluorescently labeled oligonucleotide, the fluorescently labeled base corresponding to the 395th base may exist at a position of any one of 1st to 3rd positions from the 3′ end of the P1 or P1′ fluorescently labeled oligonucleotide. Alternatively, the fluorescently labeled base may exist at the 3′ end of the P1 or P1′ fluorescently labeled oligonucleotide. Thereby, for example, the sensitivity for detecting a polymorphism is further improved. In addition, the P1 or P1′ fluorescently labeled oligonucleotide may be obtained with good productivity.

The fluorescently labeled oligonucleotide of the present invention may be a fluorescently labeled oligonucleotide in which the fluorescence intensity at the time when the oligonucleotide is hybridized to its target sequence is decreased (quenched) or increased as compared to the fluorescence intensity at the time when the oligonucleotide is not hybridized to its target sequence. In particular, the fluorescently labeled oligonucleotide of the present invention may be a fluorescently labeled oligonucleotide in which the fluorescence intensity at the time when the oligonucleotide is hybridized to its target sequence is decreased as compared to the fluorescence intensity at the time when the oligonucleotide is not hybridized to its target sequence.

A probe that uses the “fluorescence quenching phenomenon” as described above is generally referred to as a guanine quenching probe, and it is known as Q PROBE. Among such probes, an oligonucleotide which has been designed so that its 3′ or 5′ end is a cytosine (C) and which has been labeled with a fluorescent dye so that the fluorescence emission is reduced when the C at the 3′ or 5′ end comes close to a guanine (G) is especially preferable. By using such a probe, the hybridization and dissociation of the probe may be readily checked by the change in its signal.

A known detection method other than the detection method using a Q PROBE may also be applied. Examples of such a detection method include a TAQ-MAN probe method, a hybridization probe method, a molecular beacon method, and a MGB probe method.

The fluorescent dye is not particularly limited, and examples of the fluorescent dye include fluorescein, phosphor, rhodamine and polymethine dye derivatives. Examples of commercially available products of such fluorescent dyes include Pacific Blue, BODIPY FL, FluorePrime, Fluoredite, FAM, Cy3 and CyS, and TAMRA.

The detection conditions of the fluorescent-labeled oligonucleotide are not particularly limited, and may be decided, as appropriate, in accordance with the fluorescent dye to be used. For example, Pacific Blue can be detected at a detection wavelength of from 445 nm to 480 nm, TAMRA can be detected at a detection wavelength of from 585 nm to 700 nm, and BODIPY FL can be detected at a detection wavelength of from 520 nm to 555 nm.
By using a probe having such a fluorescent dye, hybridization and dissociation of the probe can be readily confirmed based on a change in fluorescence signal thereof Attachment of a fluorescent dye to the oligonucleotide may be carried out according to an ordinary method, such as a method described in JP Application No. 2002-119291.
It should be noted that, in the present invention, the same fluorescent dye may be used, or alternatively, different fluorescent dyes may be used to label one or more of the oligonucleotide.

In addition, the fluorescent-labeled oligonucleotide may have, for example, a phosphate group added to its 3′ end. Addition of a phosphate group to the 3′ end of the fluorescent-labeled oligonucleotide sufficiently suppresses elongation of the probe itself by a gene amplification reaction. As described below, a DNA for which the presence or absence of a mutation should be detected (target DNA) may be prepared using a gene amplification method such as PCR. When the fluorescent-labeled oligonucleotide that has a phosphate group added to its 3′ end is used, the amplification reaction can be carried out even in the presence of the oligonucleotide in a reaction solution of the amplification reaction. A similar effect can be obtained also by adding a labeling substance (a fluorescent dye) as described above to the 3′ end.

The above-described P1 and P1′ fluorescently labeled oligonucleotides may be used as a probe for detecting a polymorphism in the MDR1 gene, particularly the C1236T mutant-type of the MDR1 gene.

In addition, the probe for detecting a polymorphism in the MDR1 gene may be used as a probe for melting curve analysis.
The P1 fluorescently labeled oligonucleotide and P1′ fluorescently labeled oligonucleotide according to the present invention may be produced according to a conventional method known as a method for synthesizing an oligonucleotide, such as a method as described in JP Application No. 2002-119291, except that bases are used so that the base corresponding to the 395th base in the base sequence indicated in SEQ ID NO:1 is a cytosine and the base corresponding to the 395th base is labeled with a fluorescent dye.

More preferable embodiments of the P1 fluorescently labeled oligonucleotide and the P1′ fluorescently labeled oligonucleotide include the following P1-1 fluorescently labeled oligonucleotide and the following P1′-1 fluorescently labeled oligonucleotide: (P1-1) an oligonucleotide having an identity of at least 80% to a sequence complementary to a base sequence of 7 to 38 bases in length including the 395th to the 401st bases of the base sequence indicated in SEQ ID NO:1, wherein a base corresponding to the 395th base is cytosine labeled with a fluorescent dye, the oligonucleotide recognizing a polymorphism at the 401st base of SEQ ID NO:1; and
(P1′-1) an oligonucleotide which hybridizes under stringent conditions to a complementary strand of a sequence complementary to a base sequence of 7 to 38 bases in length including the 395th to the 401st bases of the base sequence indicated in SEQ ID NO:1, wherein a base corresponding to the 395th base is cytosine labeled with a fluorescent dye, the oligonucleotide recognizing a polymorphism at the 401st base of SEQ ID NO:1.
   It should be noted that “recognizing a polymorphism at the 401st base of SEQ ID NO:1” has the same meaning as “bonding to the 401st base of SEQ ID NO:1, which exhibits a polymorphism”.

<Primer>

In the below-described method of detecting a polymorphism in the MDR1 gene, primers are used in amplifying a sequence having a MDR1 gene polymorphism to be detected by a PCR method.
The primer that may be used in the present invention is not particularly limited, as long as they are capable of amplifying a nucleic acid that contains the base corresponding to the 401th base of the sequence indicated in SEQ ID NO:1, which is a desired MDR1 gene polymorphism site to be detected.

The primers used in the PCR method are not particularly restricted as long as they are capable of amplifying a base sequence containing a region to which the polymorphism detection probe according to the present invention hybridizes, and such primers can be designed as appropriate by those skilled in the art based on the base sequences indicated in SEQ ID NOs:1 to 6. The length and Tm value of the primer may be a length of from 12 mer to 40 mer and a value of from 40° C. to 70° C., or a length of from 16 mer to 30 mer and a value of from 55° C. to 60° C.

The lengths of individual primers in a primer set do not need to be the same, although the Tm values of these primers are preferably approximately the same (or the difference between the Tm values of these primers is preferably within 5° C.).

Examples of the primers that may be used for amplifying a base sequence containing a region to which the polymorphism detection probe according to the present invention used in the polymorphism detection method according to the present invention hybridizes are shown below. It is noted here that the following examples are provided for illustrative purposes only and that, therefore, the present invention is not restricted thereto.

TABLE 2

| | | Length | SEQ |
|---|---|---|---|
| Primer | Sequence (5′→3′) | (mer) | ID NO: |
| MDR1 | ccttgaagtttttttctcactcgtcctg | 28 | 7 |
| (1236)F | | | |
| MDR1 | gtctgcccactctgcaccttc | 21 | 8 |
| (1236)R | | | |

When amplifying a base sequence containing a region to which the polymorphism detection probe according to the present invention hybridizes, from the standpoints of sensitivity and efficiency, for example, the oligonucleotides shown in Table 2 may be used as a set of paired primers.

The method of detecting a polymorphism is not particularly limited, as long as it is a method in which the fluorescently labeled nucleotide as described above is used as a probe. As an example of the polymorphism detection method in which the fluorescently labeled nucleotide as described above is used as a probe, a method of detecting a polymorphism using Tm analysis is described below.

<Polymorphism Detection Method>

The method of detecting a polymorphism in the MDR1 gene according to the present invention is a method of detecting a polymorphism in the MDR1 gene which includes detecting a polymorphism in the MDR1 gene by using at least one probe for detecting a polymorphism in the MDR1 gene as described above.
The method of detecting a polymorphism of the present invention may include at least one probe for detecting a polymorphism as described above, and this may make it possible to detect a polymoiphism(s) in the MDR1 gene easily and with a high sensitivity.
In addition, the method of detecting a polymorphism according to the present invention may be employed as a method of detecting a polymorphism in various human genes, and may include the below-described processes (I) to (IV), and may include the below-described process (V). The method of detecting a polymorphism according to the present invention has a feature of using the above-described probe, and other configurations, conditions and the like are not particularly limited by the description below.

Process (I): contacting the fluorescent-labeled probe with a single-stranded nucleic acid in a sample, to obtain a hybrid.

Process (II): dissociating the hybrid by changing the temperature of the sample containing the hybrid, and measuring a change in fluorescence signal due to the dissociation of the hybrid.
Process (III): measuring a Tm value, which is the dissociation temperature of the hybrid, based on the change in fluorescence signal.
Process (IV): detecting the presence of the MDR1 polymorphism on the single-stranded nucleic acid in the sample, based on the Tm value.
Process (V): determining the abundance ratio of single-stranded nucleic acid having the polymorphism in the total single-stranded nucleic acids contained in the sample, based on the presence of the polymorphism.

Furthermore, the method according to the present invention may further include amplifying the nucleic acid before the obtainment of the hybrid in the process (I) or simultaneously with the obtainment of the hybrid in the process (I), in addition to the processes (I) to (IV) or in addition to the processes (I) to (V).

The measurement of the Tm value in the process (III) may include not only measuring the dissociation temperature of the hybrid, but also measuring the differential values of the fluorescence signal that changes according to the temperature when the hybrid is melted.

In the present invention, the nucleic acid in the sample may be a single-stranded nucleic acid or a double-stranded nucleic acid. In a case in which the nucleic acid is a double-stranded nucleic acid, the method may include, for example, melting (dissociating) the double-stranded nucleic acid in the sample into single-stranded nucleic acids by heating before being hybridized with the fluorescent-labeled probe. The dissociation of a double-stranded nucleic acid into single-stranded nucleic acids enables hybridization with the fluorescent-labeled probe.

In the present invention, the nucleic acid contained in the sample to be detected may be, for example, a nucleic acid originally contained in a biological sample, or an amplification product obtained by amplifying a region of the gene of interest that contains a mutated site(s) of the MDR1 gene by PCR or the like using a nucleic acid originally contained in a biological sample as a template with a view of improving the detection accuracy. The length of the amplification product is not particularly limited, and may be, for example, a length of from 50 mer to 1000 mer, or a length of from 80 mer to 200 mer. Furthermore, the nucleic acid in the sample may be, for example, a cDNA that has been synthesized from RNAs derived from a biological sample (e.g., total RNAs, mRNAs, etc.) by RT-PCR (Reverse Transcription PCR).

In the present invention, the addition ratio (molar ratio) of the probe according to the present invention relative to the nucleic acids in the sample is not particularly limited. The amount of the probe to be added may be, for example, no more than 1 fold (by mol) the amount of DNAs in the sample. From the viewpoint of ensuring a sufficient detection signal, the addition ratio of the probe according to the present invention to be added relative to the nucleic acids in the sample (in a molar ratio) may be 0.1 or lower.

The “nucleic acids in the sample” may be, for example, a total of nucleic acids to be detected that have the polymorphism to be detected and nucleic acids, other than the nucleic acids to be detected, that do not have the polymorphism, or a total of amplification products containing a detection target sequence having the polymorphism to be detected and amplification products containing a sequence, other than the detection target sequence, that does not have the polymorphism. Although the ratio of the nucleic acid to be detected relative to nucleic acids in the sample is usually unknown in advance, the consequential addition ratio of the probe relative to the nucleic acids to be detected (or the amplification products containing a sequence to be detected) (in a molar ratio) may be 10 or lower. The addition ratio of the probe relative to the nucleic acids to be detected (or the amplification products containing a sequence to be detected) (in a molar ratio) may be 5 or lower, or 3 or lower. The lower limit of the ratio is not particularly limited, and may be, for example, 0.001 or higher, 0.01 or higher, or 0.1 or higher.

The above-described addition ratio of the fluorescent-labeled probe according to the present invention relative to DNAs may be, for example, a molar ratio relative to double-stranded nucleic acids or a molar ratio relative to single-stranded nucleic acids.

In the present invention, the measurement of a change in the signal caused by a temperature change for determining a Tm value may be carried out by measuring the absorbance at 260 nm on the basis of the principle described above. However, the measurement may be carried out by measuring a signal which is based on a signal from the label attached to the fluorescent-labeled probe, and which varies in accordance with the degree of the formation of a hybrid of a single-stranded DNA and the probe. Therefore, the above-described fluorescent-labeled oligonucleotide may be used as the fluorescent-labeled probe. Examples of the fluorescent-labeled oligonucleotide (hereinafter sometimes collectively referred to as “fluorescent-labeled oligonucleotide”) include a fluorescent-labeled oligonucleotide of which the fluorescence intensity when the oligonucleotide is hybridized with a target sequence thereof is decreased (quenched) as compared to the fluorescence intensity when the oligonucleotide is not hybridized with the target sequence thereof, and a fluorescent-labeled oligonucleotide of which the fluorescence intensity when the oligonucleotide is hybridized with a target sequence thereof is increased as compared to the fluorescence intensity when the oligonucleotide is not hybridized with the target sequence thereof.

The former fluorescent-labeled oligonucleotide does not show a fluorescence signal or only a weak fluorescence signal when the fluorescent-labeled oligonucleotide forms a hybrid (a double-stranded DNA) with the sequence to be detected; however, the fluorescent-labeled oligonucleotide becomes to show a fluorescence signal or shows an increased fluorescence signal when the fluorescent-labeled oligonucleotide is dissociated by heating. The latter fluorescent-labeled oligonucleotide shows a fluorescence signal when the fluorescent-labeled oligonucleotide forms a hybrid (a double-stranded DNA) with the sequence to be detected; however, the fluorescent-labeled oligonucleotide shows a decreased fluorescence signal or ceases to show a fluorescent signal when the fluorescent-labeled oligonucleotide is dissociated by heating. Therefore, similar to the measurement of the absorbance at 260 nm described above, the progress of melting can be monitored, and the Tm value can be determined by detecting the change in the fluorescence signal from the fluorescent label under the conditions specific to the fluorescent label (for example, the fluorescence wavelength thereof).

The method for detecting the change in the signal based on a signal from the fluorescent dye in the polymorphism detection method according to the present invention is described below by way of specific examples. The polymorphism detection method according to the present invention has a feature of using the fluorescent-labeled polymorphism detection probe, and other processes and conditions of the method are not limited in any way.

The sample containing a nucleic acid that serves as a template for nucleic acid amplification is not particularly limited as long as the sample contains a nucleic acid, particularly the MDR1 gene. Examples of such a sample include a sample that is derived from or can be derived from any biological source, examples of which include: a tissue such as colon or lung; a hemocyte such as a leukocyte cell; whole blood; plasma; a sputum; a suspension of oral mucosa; a somatic cell of nail, hair or the like; a germ cell; milk; gascitic fluid; a paraffin-embedded tissue; gastric juice; a gastric lavage fluid; urine; peritoneal fluid; amniotic fluid; and a cell culture. The method for sampling the sample, the method for preparing the sample containing a nucleic acid, and the like are not limited, and, conventional methods known in the art may be employed therefor. A nucleic acid obtained from such a biological source may be directly used as the template, or may be used after the sample has been subjected to pretreatment that modifies the properties of the sample.

For example, in a case in which whole blood is used as the sample, the isolation of genomic DNA from the whole blood may be carried out by a conventional method known in the art. For example, a commercially available genomic DNA isolation kit (trade name: GFX GENOMIC BLOOD DNA PURIFICATION KIT, available from GE Healthcare Biosciences), etc. may be used.

Next, a fluorescent-labeled polymorphism detection probe including the fluorescent-labeled oligonucleotide is added to the sample containing an isolated genomic DNA.

The fluorescent-labeled probe may be added to a liquid sample containing an isolated genomic DNA, or may be mixed with a genomic DNA in an appropriate solvent. The solvent is not particularly limited, and examples of the solvent include conventional solvents known in the art, such as: a buffer solution such as Tris-HCl; a solvent containing at least one of KCl, MgCl₂, MgSO₄, or glycerol; and a PCR reaction solution.

The timing of adding the fluorescent-labeled probe is not particularly limited. For example, in a case in which an amplification process such as PCR described below is carried out, the fluorescent-labeled probe may be added to the PCR amplification products after the amplification process is carried out, or may be added before the amplification process is carried out.

In a case in which the fluorescent-labeled probe is added before an amplification process such as PCR is carried out, for example, a fluorescent dye or a phosphate group may have been added to the 3′ end of the probe, as described above.

The method of amplifying a nucleic acid may be, for example, a method in which a polymerase is employed. Examples thereof include a PCR method, an ICAN method, a LAMP method, and an NASBA method. In a case in which the amplification is carried out by a method in which a polymerase is employed, the amplification may be carried out in the presence of the fluorescent-labeled probe according to the present invention. Those skilled in the art would be able to easily adjust the reaction conditions of the amplification and the like in accordance with the fluorescent-labeled probe and the polymerase to be used. In a case in which the amplification is carried out in the presence of the fluorescent-labeled probe according to the present invention, a polymorphism can be detected by only analyzing the Tm value of the fluorescent-labeled probe after the amplification of the nucleic acid is carried out, and, therefore, it is not necessary to separate the amplification product after completion of the reaction. Thus, contamination by the amplification product does not occur. In addition, since the detection can be carried out by the same apparatus as the apparatus required for the amplification, conveyance of a vessel is unnecessary, and automatization of the process is facilitated.

The DNA polymerase to be used in the PCR method may be selected, without particular limitation, from DNA polymerases that are usually used for PCR. Examples of the DNA polymerase include GENE TAQ (trade name, manufactured by NIPPON GENE CO., LTD.), PRIMESTAR MAX DNA POLYMERASE (manufactured by Takara Bio Inc.), and a Taq polymerase.

The amount of the polymerase to be used is not particularly limited as long as a usually-applied polymerase concentration is provided. For example, in a case in which a Taq polymerase is used, the concentration of the Taq polymerase may be, for example, a concentration of from 0.01 U to 100 U relative to 50 μl of the reaction solution. In this range, for example, the sensitivity of the detection of polymorphism in the MDR1 gene tends to be increased

The PCR method may be carried out under the conditions appropriately selected from usually-employed conditions.

When the amplification is carried out, the amplification may be monitored using real-time PCR so that the copy number of the DNA (a sequence to be detected) contained in the sample can be measured. In other words, the proportion of probes forming hybrids is increased as the amplification of the DNA (a sequence to be detected) by PCR proceeds, thereby changing the fluorescence intensity. By monitoring the change in the fluorescence intensity, the copy number and/or the abundance ratio of the sequence to be detected (either a normal DNA or a mutant DNA) contained in the sample can be obtained.

In the polymorphism detection method according to the present invention, the fluorescent-labeled oligonucleotide and a single-stranded nucleic acid in the sample are brought into contact with each other, thereby allowing hybridization thereof. The single-stranded nucleic acid in the sample can be prepared by, for example, dissociating the PCR amplification products obtained in the above-described manner.

The heating temperature employed for dissociation of the PCR amplification products (the heating temperature in the dissociation process) is not particularly limited as long as it is a temperature at which the amplification products can be dissociated. For example, the heating temperature may be in the range of from 85° C. to 95° C. The heating time is not particularly limited, either. The heating time may be, for example, in the range of from 1 second to 10 minutes, or from 1 second to 5 minutes.

The hybridization of the dissociated single-stranded DNA and the fluorescent-labeled oligonucleotide may be carried out by, for example, decreasing, after the dissociation process, the temperature from the heating temperature employed in the dissociation process. The temperature condition for the hybridization may be, for example, in the range of from 40° C. to 50° C.

The volume and concentration of each component in the reaction solution in the hybridization process are not particularly limited. In regard to specific examples thereof, the concentration of DNAs in the reaction solution may be, for example, a concentration of from 0.01 82 M to 1 μM, or a concentration of from 0.1 μM to 0.5 μM. The concentration of the fluorescent-labeled oligonucleotide may be, for example, in a range in which the above-described addition ratio relative to DNAs is satisfied, and may be, for example, a concentration of from 0.001 μM to 10 μM, or a concentration of from 0.001 μM to 1 μM.

The resultant hybrid of the single-stranded DNA and the fluorescent-labeled oligonucleotide is gradually heated, and a change in fluorescence signal caused by the temperature increase is measured. For example, in the case of using Q PROBE, the fluorescence intensity in the state of being hybridized with the single-stranded DNA is decreased (or quenched) as compared to the fluorescence intensity in the dissociated state. Therefore, for example, the hybrid emitting decreased fluorescence or the quenched hybrid may be gradually heated, and an increase in fluorescence intensity caused by the temperature increase may be measured.

The temperature range in which the change in fluorescence intensity is measured is not particularly limited, and the initial temperature may be, for example, a temperature of from room temperature to 85° C., or a temperature of from 25° C. to 70° C. The final temperature may be, for example, a temperature of from 40° C. to 105° C. The temperature increase rate is not particularly limited, either, and may be, for example, in the range of from 0.1° C./sec to 20° C./sec, or in the range of from 0.3° C./sec to 5° C./sec.

Next, the change in the signal is analyzed to determine the Tm value. More specifically, the Tm value may be determined by calculating a differential value at each temperature (−d(Fluorescence Intensity)/dt) from the fluorescent intensity obtained, and taking the temperature at which the differential value takes the lowest value as the Tm value. The Tm value may alternatively be determined as the point at which the increase in fluorescence intensity per unit time ((Increase in Fluorescence Intensity)/t) takes the largest value. On the contrary, in a case in which a probe of which signal intensity is increased by the formation of the hybrid, rather than a quenching probe, is used as the fluorescent-labeled probe, the signal analysis and the determination of the Tm value may be carried out by measuring a decrease in fluorescence intensity.

In the present invention, a change in fluorescence signal caused by a temperature increase (preferably an increase in fluorescence intensity) may be measured while heating the hybrid as described above. However, instead of this method, the measurement of a change in signal may alternatively be carried out, for example, in the course of hybrid formation. In other words, the temperature of the sample, to which the probe has been added, may be decreased, and a change in fluorescence signal caused by the temperature decrease may be measured in the course of hybrid formation.

For example, in case in which Q PROBE is used, the fluorescence intensity is high when the probe is added to the sample since the probe is in the dissociated state. However, when the hybrid is formed by temperature decrease, the fluorescence is decreased (or quenched). Therefore, for example, a decrease in fluorescence intensity caused by temperature decrease may be measured while gradually decreasing the temperature of the heated sample.

On the other hand, in a case in which a probe of which signal is increased by hybrid formation is used, the fluorescence intensity is low (or quenched) when the probe is added to the sample since the probe is in the dissociated state. However, when the hybrid is formed by temperature decrease, the fluorescence intensity is increased. Therefore, for example, an increase in fluorescence intensity caused by temperature decrease may be measured while gradually decreasing the temperature of the sample.

Here, the polymorphism detection method according to the present invention includes detecting, in addition to a mutant-type MDR1 gene having a mutation at the 1236th base of the exon 12 (the 401st base of the base sequence indicated in SEQ ID NO:1), at least either of a mutant-type MDR1 gene having a mutation at the 3435th base of the exon 26 (the 256th base of the base sequence indicated in SEQ ID NO:15) or a mutant-type MDR1 gene having a mutation at the 2677th base of the exon 21 (the 300th base of the base sequence indicated in SEQ ID NO:16).

By this, in addition to the mutant-type of the 401st base of the base sequence indicated in SEQ ID NO:1, at least either of the mutant-type of the 256th base of the base sequence indicated in SEQ ID NO:15 or the mutant-type of the 300th base of the base sequence indicated in SEQ ID NO:16 can be further detected.

Specific means for the detection is not particularly restricted and examples thereof include the use of the probe according to the present invention which detects a polymorphism at the 401st base of the base sequence indicated in SEQ ID NO:1 in combination with at least either of a probe which detects a polymorphism corresponding to the 256th base of the base sequence indicated in SEQ ID NO:15 or a probe which detects a polymorphism corresponding to the 300th base of the base sequence indicated in SEQ ID NO:16.

The above-described probe which detects a polymorphism corresponding to the 256th base of the base sequence indicated in SEQ ID NO:15 is not particularly restricted and examples thereof include the probe described in JP Application No. 2005-287335 and a probe having the base sequence indicated in SEQ ID NO:9.

The above-described probe which detects a polymorphism corresponding to the 300th base of the base sequence indicated in SEQ ID NO:16 is not particularly restricted and examples thereof include a probe having the base sequence indicated in SEQ ID NO:10.

Further, the polymorphism detection method according to the present invention also includes the process of detecting, in addition to a polymorphism at the 401st base of the base sequence indicated in SEQ ID NO:1, at least either of a polymorphism at the 256th base of the base sequence indicated in SEQ ID NO:15 or a polymorphism at the 300th base of the base sequence indicated in SEQ ID NO:16, in the same system.

By this, in addition to a polymorphism at the 401st base of the base sequence indicated in SEQ ID NO:1, at least either of a polymorphism at the 256th base of the base sequence indicated in SEQ ID NO:15 or a polymorphism at the 300th base of the base sequence indicated in SEQ ID NO:16 can also be detected using the same system, which is more preferred from the standpoint of convenience.

The method of detecting plural polymorphisms present on different exons in the same system is not particularly restricted. For example, the method may be a method in which individual probes capable of detecting these polymorphisms are preliminarily mixed and the resulting mixture is added to a sample, or a method in which individual probes capable of detecting these polymorphisms are continuously added to a sample containing a single-stranded nucleic acid(s).

The term “system” means an independent reaction system formed with a sample containing a hybrid in which a fluorescently labeled oligonucleotide and a single-stranded nucleic acid are hybridized.

The polymorphism detection method according to the present invention may include the process of detecting, in the same system, a polymorphism corresponding to the 401st base of the base sequence indicated in SEQ ID NO:1, a polymorphism corresponding to the 256th base of the base sequence indicated in SEQ ID NO:15 and a polymorphism corresponding to the 300th base of the base sequence indicated in SEQ ID NO:16.

By this, not only can the three types of gene polymorphism be easily detected in one system, but also the later-described drug tolerance and efficacy associated with these three types of polymorphism can be more accurately estimated.

Specific means for the detection is not particularly restricted and examples thereof include the use of three types of probes in combination, which are the probe according to the present invention which detects a polymorphism at the 401st base of the base sequence indicated in SEQ ID NO:1, a probe which detects a polymorphism corresponding to the 256th base of the base sequence indicated in SEQ ID NO:15 and a probe which detects a polymorphism corresponding to the 300th base of the base sequence indicated in SEQ ID NO:16.

Here, with regard to the preferred embodiments and sequences of the respective probes, the above-described matters relating thereto are applicable.

<Method of Evaluating Drug Efficacy or Tolerance>

The method of evaluating a drug efficacy or tolerance of the present invention includes detecting a polymorphism in the MDR1 gene by the above-described polymorphism detection method, and evaluating the tolerance to a drug or the efficacy of a drug based on the results of the detection.
In the above-described polymorphism detection method, a polymorphism in the MDR1 gene may be detected with a high sensitivity and easily by using the probe in the present invention, and therefore, based on this polymorphism in the MDR1 gene, evaluation of a drug may be carried out with a high sensitivity and easily.

In addition, evaluation of the tolerance to a drug and the efficacy of a drug may be carried out based on whether a polymorphism(s) exists or not and/or based on the abundance ratio of a mutant sequence(s) and/or a normal sequence(s). The method of evaluating the efficacy of a drug of the present invention is useful in, for example, deciding whether the therapeutic strategy of a disease should be shifted so as to increase the dosage of the drug or use another therapeutic agent instead of the drug, based on whether a mutation(s) exists or not and/or based on the abundance ratio of a mutant sequence(s).

Further, specific examples of the drug to be evaluated include anticancer drugs, antihypertensive drugs, immunosuppressive agents and centrally acting drugs, and particularly include anticancer drugs.

<Reagent Kit>

The reagent kit for detecting a polymorphism according to the present invention includes the above-described polymorphism detection probe(s).
Since this reagent kit for detecting a polymorphism includes at least one of the above-described polymorphism detection probes, it tends to be able to, for example, more easily detect a polymorphism in the MDR1 gene.

In addition, the reagent kit in the present invention may further contain the above-described primer for amplifying a sequence having a MDR1 gene polymorphism to be detected. This may enable the reagent kit in the present invention to detect a polymorphism in the MDR1 gene with good accuracy.

With regard to a probe(s) and primer(s) that may be contained in the reagent kit, the above descriptions may be applied as they are.

Further, the reagent kit for detecting a polymorphism according to the present invention may preferably further include, in addition to the probe for detecting a polymorphism in the MDR1 gene, at least one probe selected from the group consisting of the above-described probe which detects a polymorphism corresponding to the 256th base of the base sequence indicated in SEQ ID NO:15 and the probe which detects a polymorphism corresponding to the 300th base of the base sequence indicated in SEQ ID NO:16.

By allowing the reagent kit for detecting a polymorphism according to the present invention to further include the above-described other probes in addition to the polymorphism detection probe for the MDR1 gene, plural polymorphisms present on plural exons can be detected easily.

In a case in which two or more types of oligonucleotide are contained as the probes, the oligonucleotides may be contained in a mixed state, or may be contained in the state of being separate from each other.

The two or more types of fluorescent-labeled oligonucleotide may be respectively labeled with fluorescent dyes having different emission wavelengths from each other.
By using the probes labeled with respectively different fluorescent dyes, detection of the signal from each fluorescent-labeled oligonucleotide can simultaneously be carried out even in a single reaction system.

Besides the probe and the primers, the reagent kit according to the present invention may further include reagents required for carrying out the nucleic acid amplification in the detection method according to the present invention. The probe, the primers and other reagents may be separately contained, or some of them may be contained in the state of a mixture.

The term “separately contained” may refer to a state in which individual reagents are separated from each other such that the non-contact state therebetween is maintained, and does not necessarily require that the individual reagents be contained in separate containers that can be independently handled.
When the reagent kit includes a primer set for amplifying a base sequence including a base at the polymorphism site (a region to which the probe can hybridize), detection of the polymorphism with higher sensitivity, for example, can be achieved.

The reagent kit according to the present invention may further include an instruction manual that describes instructions for the formation of a differential melting curve for a sample containing a nucleic acid to be detected using the MDR1 probe, and for the detection of a polymorphism in a gene-encoding base sequence through Tm value analysis based on the differential melting curve, or instructions that describes various reagents that are contained, or may additionally be contained, in the reagent kit.

### EXAMPLES

The present invention will now be described in detail by way of examples. However, the present invention is not limited to these examples in any way.

### Example 1

Using a fully-automated SNP analyzer (trade name: I-DENSY (trademark); manufactured by ARKRAY, Inc.) and a test reagent having the formulation shown in Table 3 below, Tm analysis was performed to verify the performance of the MDR1(1236) probe of a fluorescently labeled oligonucleotide (SEQ ID NO:2). Here, the artificial oligo sequences indicated in SEQ ID NOs:18 and 19 were used as templates.

TABLE 3
(Amount of reaction solution: 50 μl)
1 × Gene Taq Universal buffer
(manufactured by NIPPON GENE CO., LTD.)

| | |
|---|---|
| MDR1(1236) probe | 0.2 μM |
| Template | 0.2 μM |

The details of the probe and templates used in the above Table 3 are shown below. Here, the type of the fluorescent dye is indicated in the parentheses at the 3′-end of the probe. In the base sequence shown in Table 4, the capital letter indicates the position of the polymorphism. The same applies hereinafter.

TABLE 4

| | | |
|---|---|---|
| IMDR1(1236) | ttcaggttcagAcccttc- | SEQ ID NO: 21 |
| probe | (TAMRA) | |

TABLE 5

| Sequence (5′→3′) | Length | SEQ ID NO: |
|---|---|---|
| gatcttgaagggTctgaacctgaag | 25 | 18 |
| gatcttgaagggCctgaacctgaag | 25 | 19 |

The Tm analysis was performed by treating the reaction solution at 95° C. for 1 second and then at 40° C. for 60 seconds and subsequently measuring the change in the fluorescence intensity over time during a period in which the temperature of the resultant was increased from 40° C. to 75° C. at a rate of 1° C./3 seconds. Here, the excitation wavelength and the detection wavelength were set in the range of 520 nm to 555 nm and 585 nm to 700 nm, respectively, to measure the changes in the fluorescence intensity originated from the respective fluorescently labeled probes.

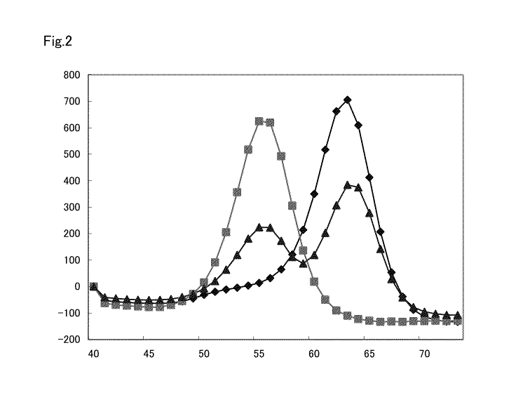

The Tm analysis yielded FIG. 2 showing the changes in the fluorescence value of the respective probes. In FIG. 2, the ordinate indicates the change in the fluorescence intensity per unit time (increase in the d-fluorescence intensity/t), and the abscissa indicates the temperature (° C.). In FIG. 2, the pattern indicated with lozenges represents the results obtained by using, as a template, the artificial oligo sequence indicated in SEQ ID NO:19 having the same base sequence as indicated in SEQ ID NO:1 except that the 401st base (Y) is C. The pattern indicated with squares represents the results obtained by using, as a template, the artificial oligo sequence indicated in SEQ ID NO:18 having the same base sequence as indicated in SEQ ID NO:1 except that the 401st base (Y) is T, and the pattern indicated with triangles represents the results obtained by using, as a template, a 1:1 mixture of the artificial oligo sequences indicated in SEQ ID NOs:18 and 20.

From the results shown in FIG. 2, it was proven that, by using the fluorescently labeled oligonucleotide according to the present invention as a probe, a polymorphism at the 401st base of the base sequence indicated in SEQ ID NO:1 can be detected even when a sequence having the same bases as indicated in SEQ ID NO:1 except that the 401st base (Y) is C coexists with a sequence having the same bases as indicated in SEQ ID NO:1 except that the 401st base (Y) is T. Further, the Tm value was 60° C. when the artificial oligo sequence indicated in SEQ ID NO:19 was used as a template and 56° C. when the artificial oligo sequence indicated in SEQ ID NO:18 was used as a template.

### Example 2

Using a fully-automated SNP analyzer (trade name: I-DENSY (trademark); manufactured by ARKRAY, Inc.) and a reaction solution having the formulation shown in Table 6 or 7 below, PCR and Tm analysis were performed.

TABLE 6
<<When a purified human genome was used as a template>>

| (Amount of reaction solution: 50 μl) | | |
|---|---|---|
| 1 × PCR buffer | | |
| dNTP | 0.2 | mM |
| MgCl2 | 1.5 | mM |
| Taq polymerase (manufactured by ARKRAY, Inc.) | 1.88 | U/test |
| MDR1 exon26 probe | 0.2 | μM |
| MDR1 exon21 probe 2 | 0.2 | μM |
| MDR1(1236) probe | 0.2 | μM |
| MDR1 exon21-F | 1 | μM |
| MDR1 exon21-R | 0.2 | μM |
| NDR1 exon26-F | 1 | μM |
| MDR1 exon26-R | 0.2 | μM |
| MDR1(1236)F | 1 | μM |
| MDR1(1236)R | 0.2 | μM |
| Purified human genome | 100 | copies |

Human genome purified from whole blood was used

TABLE 7
<<When whole blood was used as template>>

| (Amount of reaction solution: 50 μl) | | |
|---|---|---|
| 1 × PCR buffer | | |
| dNTP | 0.2 | mM |
| MgCl2 | 1.5 | mM |
| Taq polymerase (manufactured by ARKRAY, Inc.) | 1.88 | U/test |
| MDR1 exon26 probe | 0.2 | μM |
| MDR1 exon21 probe 2 | 0.2 | μM |
| MDR1(1236) probe | 0.2 | μM |
| MDR1 exon21-F | 1 | μM |
| MDR1 exon21-R | 0.2 | μM |
| NDR1 exon26-F | 1 | μM |
| MDR1 exon26-R | 0.2 | μM |
| MDR1(1236)F | 1 | μM |
| MDR1(1236)R | 0.2 | μM |
| Pre-treated whole blood | 4 | μl |

The PCR was performed by treating the reaction solution at 95° C. for 1 second and then repeating 50 cycles of 95° C. for 1 second and 57° C. for 30 seconds.

The Tm analysis was performed after the PCR by treating the reaction solution at 95° C. for 1 second and then at 40° C. for 60 seconds and subsequently measuring the change in the fluorescence intensity over time during a period in which the temperature of the resultant was increased from 40° C. to 75° C. at a rate of 1° C./3 seconds.

As a polymorphism detection probe, a fluorescently labeled oligonucleotide having the same sequence as indicated in SEQ ID NO:2 except that the 3′-end is labeled with a fluorescent dye (TAMRA) (MDR1(1236) probe), a fluorescently labeled oligonucleotide having the same sequence as indicated in SEQ ID NO:9 except that the 5′-end is labeled with a fluorescent dye (PACIFIC BLUE) (MDR1 exon 26 probe) and a fluorescently labeled oligonucleotide having the same sequence as indicated in SEQ ID NO:10 except that the 5′-end is labeled with a fluorescent dye (BODIPY FL) (MDR1 exon 21 probe2) were employed.

The details of the MDR1 exon 26 probe and the MDR1 exon 21 probe are shown below.

TABLE 8

| | | SEQ |
|---|---|---|
| Probe | Sequence (5′→3′) | ID NO: |
| MDR1 | (PACIFIC BLUE)-ctgccctcacAatctcttc-P | 9 |
| exon26 | | |
| probe | | |
| MDR1 | (BODIPY FL)-cccagAaccttctagttc-P | 10 |
| exon21 | | |
| probe2 | | |

Here, in the fluorescent dye, PACIFIC BLUE, the excitation wavelength is 365 nm to 415 nm, and the detection wavelength is 445 nm to 480 nm. In the fluorescent dye, BODIPY FL, the excitation wavelength is 420 nm to 485 nm, and the detection wavelength is 520 nm to 555 nm. In the fluorescent dye, TAMRA, the excitation wavelength is 520 nm to 555 nm, and the detection wavelength is 585 nm to 700 nm (the same applies hereinafter). Based on these wavelengths, the changes in the fluorescence intensity originated from the respective fluorescently labeled probes were measured.

Among those primers that were used, the details of the primers other than the MDR1(1236)F indicated in SEQ ID NO:7 and the MDR1(1236)R indicated in SEQ ID NO:8 are shown below.

TABLE 9

| Primer | Sequence (5′→3′) | SEQ ID NO: |
|---|---|---|
| MDR1 | aaatgttgtctggacaagcactg | 11 |
| exon21-F | | |
| MDR1 | aattaatcaatcatatttagttt | 12 |
| exon21-R | gactcac | |
| MDR1 | actgcagcattgctgagaac | 13 |
| exon26-F | | |
| MDR1 | cagagaggctgccacatgctc | 14 |
| exon26-R | | |

As a template, whole blood and human genome purified from whole blood by a conventional method were employed.

The whole blood was prepared in the following manner.

To 70 μl of diluent 1, 10 μl of whole blood was added and mixed well, and 10 μl of the resulting mixture was then added to 70 μl of diluent 2. By heating 17 μl of the thus obtained mixture at 95° C. for 10 minutes, 4 μl of pre-treated whole blood was obtained. This was used as a template per one test.

TABLE 10

| Diluent 1 | | | Diluent 2 | | |
|---|---|---|---|---|---|
| Tris-HCl (pH 8.0) | 10 | mM | Tris-HCl (pH 8.0) | 10 | mM |
| EDTA (pH 8.0) | 0.1 | mM | 500 mM EDTA (pH 8.0) | 0.1 | mM |

| | |
|---|---|
| SDS | 0.30% |

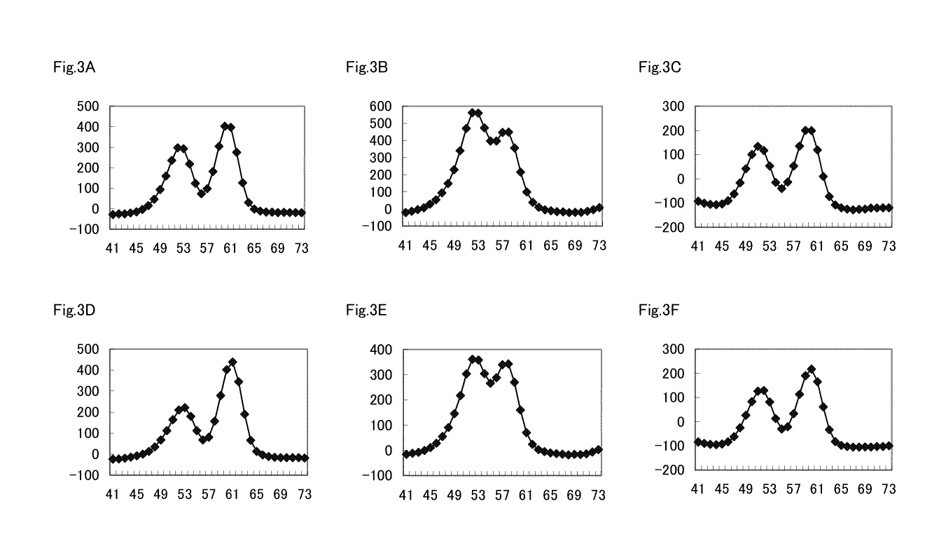

The Tm analysis yielded FIG. 3 showing the changes in the fluorescence value of the respective probes.

FIGS. 3(A) to (C) show the results obtained by using the purified human genome as a sample.

FIGS. 3(D) to (F) show the results obtained by using the whole blood as a sample.

Further, FIGS. 3(A) and (D) show the presence or absence of a polymorphism corresponding to the 401st base of the base sequence indicated in SEQ ID NO:1, and FIGS. 3(B) and (E) show the presence or absence of a polymorphism corresponding to the 256th base of the base sequence indicated in SEQ ID NO:15. FIGS. 3(C) and (F) show the presence or absence of a polymorphism corresponding to the 300th base of the base sequence indicated in SEQ ID NO:16.

In these figures, the ordinate indicates the change in the fluorescence intensity per unit time (increase in the d-fluorescence intensity/t), and the abscissa indicates the temperature (° C.).

As a result, it was proven that, by using the fluorescently labeled oligonucleotide according to the present invention as a probe, a polymorphism at the 401st base of the base sequence indicated in SEQ ID NO:1 can be easily detected in the same system simultaneously with the presence or absence of a polymorphism corresponding to the 256th base of the base sequence indicated in SEQ ID NO:15 and a polymorphism corresponding to the 300th base of the base sequence indicated in SEQ ID NO:16.

### Comparative Example 1

Using a fully-automated SNP analyzer (trade name: I-DENSY (trademark); manufactured by ARKRAY, Inc.) and a test reagent having the formulation shown in Table 11 below, Tm analysis was performed. Here, the fluorescently labeled oligonucleotide indicated in SEQ ID NO:17 was used as a probe, and the artificial oligo sequences indicated in SEQ ID NOs:20 and 21 were used as templates.

TABLE 11
(Amount of reaction solution: 50 μl)
1 × Gene Taq Universal buffer (manufactured
by NIPPON GENE CO., LTD.)

| | |
|---|---|
| Probe | 0.2 μM |
| Template | 0.2 μM |

The details of the probe and templates used in the above Table 11 are shown below. Here, the type of the fluorescent dye is indicated in the parentheses at the 3′-end of the probe.

TABLE 12

| | Length | Tm value | | Δ value between | SEQ |
|---|---|---|---|---|---|
| Sequence (5′→3′) | (mer) | T | C | C and T | ID NO: |
| tgaagggTctgaacc-(TAMRA) | 15 | 45 | 39 | 6 | 17 |

TABLE 13

| Sequence (5′→3′) | Length | SEQ ID NO: |
|---|---|---|
| cttcaggttcagAcccttcaagatc | 25 | 20 |
| cttcaggttcagGcccttcaagatc | 25 | 21 |

The Tm analysis was performed by treating the reaction solution at 95° C. for 1 second and then at 40° C. for 60 seconds and subsequently measuring the change in the fluorescence intensity over time during a period in which the temperature of the resultant was increased from 40° C. to 75° C. at a rate of 1° C./3 seconds. As the fluorescent dye, TAMRA was employed.

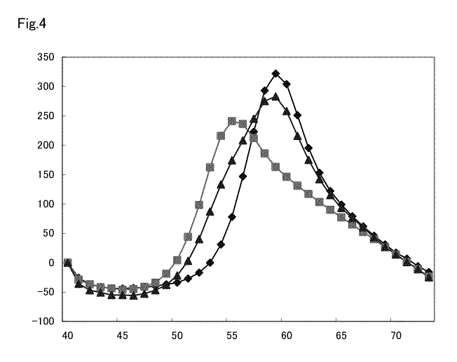

The Tm analysis yielded FIG. 4 showing the changes in the fluorescence value of the respective probes. In FIG. 4, the ordinate indicates the change in the fluorescence intensity per unit time (increase in the d-fluorescence intensity/t), and the abscissa indicates the temperature (° C.). In FIG. 4, the pattern indicated with lozenges represents the results obtained by using, as a template, the artificial oligo sequence indicated in SEQ ID NO:21 having the same base sequence as indicated in SEQ ID NO:1 except that the 401st base (Y) is C. The pattern indicated with squares represents the results obtained by using, as a template, the artificial oligo sequence indicated in SEQ ID NO:20 having the same base sequence as indicated in SEQ ID NO:1 except that the 401st base (Y) is T, and the pattern indicated with triangles represents the results obtained by using, as a template, a 1:1 mixture of the artificial oligo sequences indicated in SEQ ID NOs:20 and 22.

According to the results shown in FIG. 4, when the sequence having the same bases as indicated in SEQ ID NO:1 except that the 401st base (Y) is C coexisted with the sequence having the same bases as indicated in SEQ ID NO:1 except that the 401st base (Y) is T, only one detection peak was obtained, and a polymorphism could not be detected.

It is noted here that the fluorescently labeled oligonucleotide indicated in SEQ ID NO:17 which was used in Comparative Example 1 was presented merely as a representative example of undetectable probes that exist in large numbers.

From the above-described results, it was demonstrated that, by the present invention, a polymorphism in the MDR1 gene can be detected easily with high sensitivity.

1. A probe for detecting a polymorphism in the MDR1 gene, which is a fluorescently labeled oligonucleotide selected from the group consisting of the following P1 and P1′:
(P1) an oligonucleotide having an identity of at least 80% to a sequence complementary to a base sequence of 7 to 38 bases in length including the 395th to the 401st bases of the base sequence indicated in SEQ ID NO:1, wherein a base corresponding to said 395th base is cytosine labeled with a fluorescent dye; and
(P1′) an oligonucleotide which hybridizes under stringent conditions to a complementary strand of a sequence complementary to a base sequence of 7 to 38 bases in length including the 395th to the 401st bases of the base sequence indicated in SEQ ID NO:1, wherein a base corresponding to said 395th base is cytosine labeled with a fluorescent dye.

2. The probe according to claim 1, which is at least one fluorescently labeled oligonucleotide selected from the group consisting of the following P1-1 and P1′-1:
(P1-1) an oligonucleotide having an identity of at least 80% to a sequence complementary to a base sequence of 7 to 38 bases in length including the 395th to the 401st bases of the base sequence indicated in SEQ ID NO:1, wherein a base corresponding to said 395th base is cytosine labeled with a fluorescent dye, said oligonucleotide recognizing a polymorphism at the 401st base of SEQ ID NO:1; and
(P1′-1) an oligonucleotide which hybridizes under stringent conditions to a complementary strand of a sequence complementary to a base sequence of 7 to 38 bases in length including the 395th to the 401st bases of the base sequence indicated in SEQ ID NO:1, wherein a base corresponding to said 395th base is cytosine labeled with a fluorescent dye, said oligonucleotide recognizing a polymorphism at the 401st base of SEQ ID NO:1.

3. The probe according to claim 1, wherein said P1 fluorescently labeled oligonucleotide or P1′ fluorescently labeled oligonucleotide comprises a base corresponding to said 395th base labeled with a fluorescent dye at any one of the first to the third positions from the 3′-end.

4. The probe according to claim 1, wherein said P1 fluorescently labeled oligonucleotide or P1′ fluorescently labeled oligonucleotide comprises a base corresponding to said 395th base labeled with a fluorescent dye at the 3′-end.

5. The probe according to claim 1, wherein said P1 fluorescently labeled oligonucleotide or P1′ fluorescently labeled oligonucleotide emits fluorescence when it is not hybridized to a target sequence, and the fluorescence intensity of said P1 fluorescently labeled oligonucleotide or P1′ fluorescently labeled oligonucleotide when hybridized to said target sequence is decreased or increased as compared to when not hybridized to said target sequence.

6. The probe according to claim 5, wherein said P1 fluorescently labeled oligonucleotide or P1′ fluorescently labeled oligonucleotide emits fluorescence when it is not hybridized to a target sequence, and the fluorescence intensity of said P1 fluorescently labeled oligonucleotide or P1′ fluorescently labeled oligonucleotide when hybridized to said target sequence is decreased as compared to when not hybridized to said target sequence.

7. The probe according to claim 1, wherein said P1 fluorescently labeled oligonucleotide or P1′ fluorescently labeled oligonucleotide has a length of 7 to 28 bases.

8. The probe according to claim 1, wherein said P1 fluorescently labeled oligonucleotide or P1′ fluorescently labeled oligonucleotide has a length of 7 to 18 bases.

9. The probe according to claim 1, which is a probe for melting curve analysis.

10. A method of detecting a polymorphism in the MDR1 gene, the method comprising the processes of:
(I) bringing the probe according to claim 1 into contact with a single-stranded nucleic acid contained in a sample to hybridize said fluorescently labeled oligonucleotide to said single-stranded nucleic acid, thereby obtaining a hybrid;
(II) dissociating said hybrid by changing the temperature of said sample containing said hybrid to measure the change in the fluorescence signal caused by dissociation of said hybrid;
(III) determining a Tm value, which is the dissociation temperature of said hybrid, based on said change in the fluorescence signal; and
(IV) based on said Tm value, detecting the presence of a polymorphism of the MDR1 gene on said single-stranded nucleic acid in said sample.

11. The method according to claim 10, which further comprises the process of amplifying said nucleic acid prior to or simultaneously with said process (I) of obtaining a hybrid.

12. The method according to claim 10, which further comprises the process of detecting, in the same system, at least either of a polymorphism corresponding to the 256th base of the base sequence indicated in SEQ ID NO:15 or a polymorphism corresponding to the 300th base of the base sequence indicated in SEQ ID NO:16.

13. The method according to claim 10, which comprises the process of detecting, in the same system, a polymorphism corresponding to the 401st base of the base sequence indicated in SEQ ID NO:1, a polymorphism corresponding to the 256th base of the base sequence indicated in SEQ ID NO:15 and a polymorphism corresponding to the 300th base of the base sequence indicated in SEQ ID NO:16.

14. A method of evaluating efficacy of a drug, which comprises the processes of:
detecting a polymorphism in the MDR1 gene by the method of detecting a polymorphism according to claim 10; and
determining the tolerance to said drug or the efficacy of said drug based on the presence or absence of detected polymorphism.

15. A reagent kit for detecting a polymorphism in the MDR1 gene, which comprises the probe according to claim 1.

16. The reagent kit according to claim 15, which further comprises a primer for amplifying a base sequence containing a region to which said probe hybridizes.

17. The reagent kit according to claim 15, which further comprises a probe for detecting a polymorphism corresponding to the 256th base of the base sequence indicated in SEQ ID NO:1 and a probe for detecting a polymorphism corresponding to the 300th base of the base sequence indicated in SEQ ID NO:16.


SEQUENCE LISTING (21 sequences)

SEQ ID NO: 1 - DNA, 801 bp, Artificial Sequence
  Exon 12 of MDR1 gene having a polymorphism at the 1236th base of exon 12
ttgtccagttaagtcaatttctttgtcactttatccagctctccacaaaatatcactaaaagtagttattgtaacctagtaatctcttaaaatttgattctgtttagaagccaagtattgacagctattcgaagagtgggcacaaaccagataatattaagggaaatttggaattcagaaatgttcacttcagttacccatctcgaaaagaagttaaggtacagtgataaatgattaatcaacaattaatctattgaatgaagagtttctgatgttttcttgtagagattataaaaaagtgcatgtatatttaaacctagtgaacagtcagttcctatatcctgtgtctgtgaattgccttgaagtttttttctcactcgtcctggtagatcttgaagggyctgaacctgaaggtgcagagtgggcagacggtggccctggttggaaacagtggctgtgggaagagcacaacagtccagctgatgcagaggctctatgaccccacagaggggatggtgagatgacccatgcgagctagaccctgcggtgatcagcagtcacattgcacatctttctgatgttgccctttcaattacaaatgtatgaaagtcacacttactttttattccaggtcagtgttgatggacaggatattaggaccataaatgtaaggtttctacgggaaatcattggtgtggtgagtcaggaacctgtattgtttgccaccacgatagctgaaaacattcgctatggccgtgaaaatgtcaccatggatgagattgagaaagctgtcaaggaagc

SEQ ID NO: 2 - DNA, 18 bp, Artificial Sequence
  Probe
ttcaggttcagacccttc

SEQ ID NO: 3 - DNA, 23 bp, Artificial Sequence
  Probe
gcaccttcaggttcagacccttc

SEQ ID NO: 4 - DNA, 28 bp, Artificial Sequence
  Probe
actctgcaccttcaggttcagacccttc

SEQ ID NO: 5 - DNA, 38 bp, Artificial Sequence
  Probe
ccgtctgcccactctgcaccttcaggttcagacccttc

SEQ ID NO: 6 - DNA, 48 bp, Artificial Sequence
  Probe
accagggccaccgtctgcccactctgcaccttcaggttcagacccttc

SEQ ID NO: 7 - DNA, 28 bp, Artificial Sequence
  Primer
ccttgaagtttttttctcactcgtcctg

SEQ ID NO: 8 - DNA, 21 bp, Artificial Sequence
  Primer
gtctgcccactctgcaccttc

SEQ ID NO: 9 - DNA, 19 bp, Artificial Sequence
  Probe
ctgccctcacaatctcttc

SEQ ID NO: 10 - DNA, 18 bp, Artificial Sequence
  Probe
cccagaaccttctagttc

SEQ ID NO: 11 - DNA, 23 bp, Artificial Sequence
  Primer
aaatgttgtctggacaagcactg

SEQ ID NO: 12 - DNA, 30 bp, Artificial Sequence
  Primer
aattaatcaatcatatttagtttgactcac

SEQ ID NO: 13 - DNA, 20 bp, Artificial Sequence
  Primer
actgcagcattgctgagaac

SEQ ID NO: 14 - DNA, 21 bp, Artificial Sequence
  Primer
cagagaggctgccacatgctc

SEQ ID NO: 15 - DNA, 511 bp, Artificial Sequence
  Exon 26 of MDR1 gene having a polymorphism at the 3435th base of exon 26
ctcacagtaacttggcagtttcagtgtaagaaataatgatgttaattgtgctacattcaaagtgtgctggtcctgaagttgatctgtgaactcttgttttcagctgcttgatggcaaagaaataaagcgactgaatgttcagtggctccgagcacacctgggcatcgtgtcccaggagcccatcctgtttgactgcagcattgctgagaacattgcctatggagacaacagccgggtggtgtcacaggaagagatygtgagggcagcaaaggaggccaacatacatgccttcatcgagtcactgcctaatgtaagtctctcttcaaataaacagcctgggagcatgtggcagcctctctggcctatagtttgatttataaggggctggtttcccagaagtgaagagaaattagcaaccaaatcacacccttacctgtatacaagcatctggccacacttcctgtttgggttagttgttacctttacctgatcacctgaccctccttgtgag

SEQ ID NO: 16 - DNA, 600 bp, Artificial Sequence
  Exon 21 of MDR1 gene having a polymorphism at the 2677th base of exon 21
taaaataatgaatatagtctcatgaaggtgagttttcagaaaatagaagcatgagttgtgaagataatatttttaaaatttctctaatttgttttgttttgcaggctrtaggttccaggcttgctgtaattacccagaatatagcaaatcttgggacaggaataattatatccttcatctatggttggcaactaacactgttactcttagcaattgtacccatcattgcaatagcaggagttgttgaaatgaaaatgttgtctggacaagcactgaaagataagaaagaactagaaggtdctgggaaggtgagtcaaactaaatatgattgattaattaagtagagtaaagtattcyaatcagtgttattttgtyactccctactgcttactatgctctaagaatgtgtttataaccattcctcaaagcaatctttttcatgcttattcagtaaattagaaacttacagaaagtagcaaagccagttcttggactcaaaaactgataattaactttaacagactttttcagttttcaggccattgtcttcacactgttcttccttcctccccactttcctccttcccttagttatkttct

SEQ ID NO: 17 - DNA, 15 bp, Artificial Sequence
  Probe
tgaagggtctgaacc

SEQ ID NO: 18 - DNA, 25 bp, Artificial Sequence
  Template oligo sequence
gatcttgaagggtctgaacctgaag

SEQ ID NO: 19 - DNA, 25 bp, Artificial Sequence
  Template oligo sequence
gatcttgaagggcctgaacctgaag

SEQ ID NO: 20 - DNA, 25 bp, Artificial Sequence
  Template oligo sequence
cttcaggttcagacccttcaagatc

SEQ ID NO: 21 - DNA, 25 bp, Artificial Sequence
  Template oligo sequence
cttcaggttcaggcccttcaagatc